(19) United States
(12) Patent Application Publication
(10) Pub. No.: US 2005/0021080 A1
(43) Pub. Date: Jan. 27, 2005

(54) Inflatable apparatus for accessing a body cavity and methods of making

(76) Inventors: Gerald Feuer, Atlanta, GA (US); Michael Hogendijk, Palo Alto, CA (US); Gerald J. Sanders, Sonoma, CA (US)

Correspondence Address: David E. Heisey, Esq.;Luce, Forward, Hamilton & Scripps LLP, 11988 El Camino Real, Suite 200, San Diego, CA, 92130, US

(21) Appl. No.: 10/719,542

(22) Filed: Nov. 20, 2003

Related U.S. Application Data
(60) Provisional application No. 60/490,086, filed on Jul. 24, 2003.

(51) Int. Cl. A61B 17/28

(52) U.S. Cl. 606/207

(57) ABSTRACT

Apparatus is provided for accessing a body cavity comprising a tubular member formed by everting a length of material upon itself to form a tubular annulus. The tubular member has a deflated insertion configuration and an inflated expanded configuration that facilitates viewing within the body cavity. The tubular member may include proximal and distal cuffs that facilitate placement of the apparatus within a body cavity or organ, and an inflation lumen that enhances rigidity of the device in the deflated configuration, thereby assisting insertion into the body cavity.

### REFERENCE TO RELATED APPLICATIONS

This application claims the benefit of U.S. Provisional Patent Application Ser. No. 60/490,086, filed Jul. 24, 2003 and incorporated herein by reference in its entirety.

### FIELD OF THE INVENTION

The present invention relates to methods and apparatus for accessing a body cavity, and more particularly, to methods and apparatus for gaining access to the female urogenitary tract.

### BACKGROUND OF THE INVENTION

Examination of the vagina and its associated anatomy is typically performed using a speculum, which provides access to the vagina by dilating the vaginal canal and then holding it in an expanded state. As currently used, a conventional speculum consists of a pair of metal jaws that are inserted into the vaginal canal and then actuated to expand the canal. For most patients, insertion and operation of the speculum is uncomfortable and may cause the patient to become tense, thus making a thorough examination difficult, if not impossible.

Speculums having inflatable exterior walls have been developed, such as described in U.S. Pat. No. 5,716,329 to Dieter. The speculum described in that patent includes a rigid, interior wall and an inflatable exterior wall that may be inflated with fluid after insertion to alleviate discomfort associated with expansion of the vaginal canal. The device described in that patent, however, is fairly complicated and because it combines both reusable and disposable components, may not be commercially practicable.

In view of the low cost needed to have a commercially viable disposable speculum product, others have attempted to develop speculums that comprise inflatable sacs or ribs, such as described in International Patent Publication No. WO97/24975 and Dutch Patent No. 9100599. The products described in these publications and patent's do not appear to possess sufficient expansile strength for practical use, however.

In view of the aforementioned drawbacks of previously known devices, it would be desirable to provide methods and apparatus for accessing a body cavity that is small, easy to insert into the body cavity and comfortable once inserted and actuated within the body cavity.

It further would be desirable to provide methods and apparatus for accessing a body cavity that provides sufficient strength to expand the body cavity while using low-cost materials that permit the apparatus to be discarded after a single use.

### SUMMARY OF THE INVENTION

In view of the foregoing, it is an object of the present invention to provide an apparatus for accessing a body cavity that is small, easy to insert into the body cavity and comfortable once inserted and actuated within the body cavity.

It is another object of this invention to provide methods and apparatus for accessing a body cavity that provide sufficient strength to expand the body cavity while using low-cost materials that permit the apparatus to be discarded after a single use.

In accordance with the principles of the present invention, apparatus is provided for accessing a body cavity that comprises an inflatable body having a main body portion, a distal cuff and a proximal cuff. The inflatable body is inserted into the body cavity in a deflated configuration and then inflated to an expanded configuration, thereby expanding the walls of the body cavity. The inflatable body comprises interior and exterior walls that are joined at a plurality of contact points, wherein the contact points are arranged in a substantially uniform pattern to permit substantially uniform pressure distribution within the inflatable body in the expanded configuration.

Preferably, the apparatus is formed by everting a length of material upon itself to form a tubular annulus. The tubular member has a deflated insertion configuration and an inflated expanded configuration that facilitates viewing within the body cavity. The single piece of material preferably is locally thicker at its ends than within the main body portion, thus forming distal and proximal cuffs in the expanded configuration. In addition, the inflatable body preferably has a single seam disposed along the proximal cuff end, while the distal cuff is configured to engage an interior plane of a patient's uterus.

In a preferred embodiment, the apparatus includes an inflation lumen disposed along the entire length of an inflatable body. The inflation lumen preferably comprises a central spine that enhances stiffness and facilitates insertion into the body cavity. In addition, the inflation lumen may be formed in a substantially helical configuration and include a plurality of small holes disposed along its length to provide a more uniform inflation of the inflatable body.

According to some embodiments, the apparatus includes an inflatable body and a handle assembly attached to the inflatable body to facilitate insertion of the inflatable body into the body cavity. The inflatable body has a deflated configuration for insertion into the body cavity and an expanded configuration when pressurized.

According to other embodiments, the apparatus includes an inflatable body and a modular handle assembly attached to the inflatable body to facilitate insertion and manipulation of the inflatable body within the body cavity. The modular handle assembly comprises an intravaginal tongue portion and a detachable gripping portion for holding and manipulating the tongue portion.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a side-sectional view of apparatus of the present invention in a deflated configuration;

FIG. 2 is a side-sectional view of the apparatus of FIG. 1 in an expanded configuration;

FIG. 3A is a side-sectional view of the apparatus of FIG. 2 further depicting a plurality of contact points, while FIG. 3B is a cross-sectional view of the device of FIG. 3A taken along line 3B-3B;

FIGS. 4A and 4B are end views of the apparatus of FIG. 2;

FIGS. 5A-5C are side views depicting a method of making the device of FIG. 2, while FIG. 5D is a cross-sectional view depicting an alternative design of the device;

FIGS. 6A and 6B are side-sectional views of the device of FIG. 2 including means to deploy the device to the expanded configuration.

FIGS. 7A and 7B are side and perspective views, respectively, of a handle assembly configured for use with the apparatus of the present invention;

FIG. 8 is a side view of the handle assembly of FIGS. 7A and 7B in use with the apparatus of FIG. 2;

FIG. 9 is a side-sectional view of the device of FIG. 8 within a patient's vaginal canal;

FIG. 10A is a side view of an alternative handle assembly configured for use with the apparatus of the present invention, while FIGS. 10B and 10C are cross-sectional views of the handle assembly of FIG. 10A taken along lines 10B-10B and 10C-10C, respectively; configured for use with the apparatus of the present invention;

FIGS. 11A-11C are, respectively, a side view of a component of the handle assembly of FIG. 10A, a cross-sectional view of the component of FIG. 11A taken along line 11B-11B, and a side-sectional view of the component of FIG. 11A in use with the apparatus of FIG. 2; and

FIG. 12 is a side-sectional view of an alternative embodiment of the device of FIG. 11C.

### DETAILED DESCRIPTION OF THE INVENTION

Referring to FIGS. 1-3, the present invention provides low-cost single-use disposable apparatus for expanding a body cavity, such as the vaginal canal. Apparatus 10 comprised inflatable polymeric body 11 that assumes a substantially tubular shape when inflated from a deflated configuration (FIG. 1) to an expanded configuration (FIG. 2). In the expanded configuration, inflatable body 11 forms annular main body portion 12 defining central lumen 13, distal cuff 15 and proximal cuff 16. Inflation lumen 17 may be disposed within the thickness of the wall of main body portion 12, or alternatively may be disposed within central lumen 13. Inflation lumen 17 communicates with the interior of main body portion 12 to permit body 11 to be inflated and deflated. Cuffs 15 and 16 preferably include an outer diameter that is greater that the outer diameter of the main body portion.

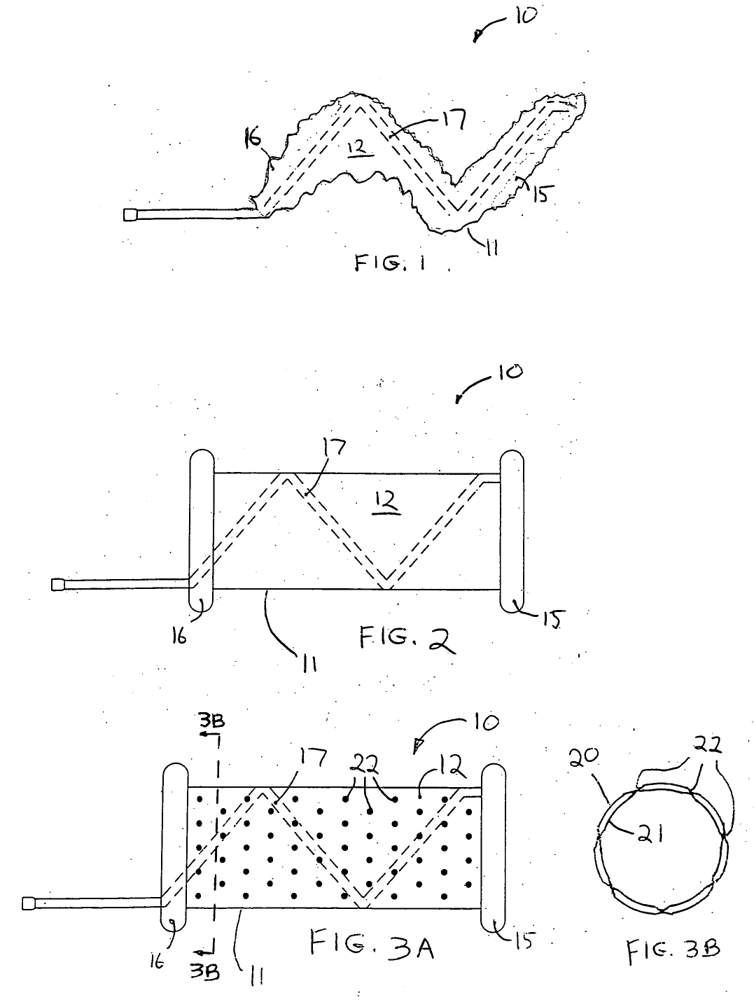

Referring to FIGS. 3A and 3B, inflatable body 11 preferably comprises a polymeric, latex-free material and is formed so that exterior wall 20 is joined to interior wall 21 at plurality of pillow-like quilted contact points 22. Preferably, contact points 22 are arranged in a uniform pattern to allow for substantially uniform circumferential pressure distribution within the inflatable body 11, especially in the expanded configuration.

Inflatable body 11 preferably comprises a single piece of material that is everted onto itself to form a double-layer tubular annulus that is approximately half as long as the original piece of material. More preferably, the material includes areas of increased thickness that are disposed at the distal and proximal ends of the tubular annulus when everted, so that when the device is inflated, the areas of increased thickness form distal and proximal cuffs 15 and 16. In alternative embodiments, cuffs 15 and 16 may be preformed, for example, by blowing the material into an appropriately shaped mold, or they may be separately formed and then attached to main body portion 12 using suitable adhesives or heat welding.

Referring to FIGS. 5A-5C, a method of making the apparatus of FIGS. 1-3 is described. In FIG. 5A, a tubular length of material 25 having first end 26, second end 27, and thickness T and length L is provided. In FIG. 5B, the length of material is everted by pulling first end 26 over the tube toward second end 27.

Referring to FIG. 5C, everting tubular material forms a double-layer tubular annulus having aproximately one-half of the original length. Inflatable body 11 thus has exterior wall 20, interior wall 21, closed end 26′ and open end 27′. Next, seam 30 is formed at open end 27′ using suitable adhesives, ultrasonic welding or heat welding, to seal the inflatable body 11. Contact points 22 may be formed between walls 20 and 21, for example, by heat welding the walls to each other.

Optionally, exterior wall 20 may be provided with coating 29, for example, a drug coating containing an antibiotic for topical distribution. Alternatively, coating 29 may be a gene or protein coating. By providing the coating on the exterior wall, the antibiotic, gene or protein may be delivered directly to the vaginal wall during examination and treatment. By way of example, coating 29 may contain Novocain, contraceptives, fertilization preparations, coagulants and various genes and proteins. Depending upon the pharmicokinetics of various drugs, genes and proteins and how they are absorbed in the vagina, coating 29 may contain more than one drug to be delivered into the vagina.

To facilitate delivery of the drug, gene or protein, features or patterns may be provided on the exterior wall. Alternatively, coating 29 may be lubricious and become slippery when exposed to water, thus reducing friction encountered during insertion of the device. As a further alternative, the apparatus may be pre-soaked in warm water prior to insertion to reduce patient discomfort, as the inflatable body is expected to retain some of the heat from the warm water.

Referring to FIG. 5D, according to some embodiments, inflatable body 11 is formed as a three-layered tube including interior layer 31, middle layer 32 and exterior layer 36 including plurality of micro-perforations 38. The area between the interior layer and the middle layer forms inflation pocket 32a that is filled with gas or fluid to expand inflatable body 11. The area between the middle layer and the exterior layer forms pocket 36a, which is adapted to be filled with drugs, proteins or genes in a liquid or gel form. When the inflation pocket is expanded inside a patient's vagina, the drugs, proteins or-genes within pocket 36a are forced through micro-perforations 38a and delivered to the vaginal wall.

According to some embodiments, coating 29 or pocket 36a may include medications for treating yeast infections. Some suitable medications include terazol, diflucan, monistat and gynazole. According to other embodiments, coating 29 or pocket 36a may include medications for treating bacterial infections. Some suitable medications include flagy and cleocin.

Advantageously, a device constructed in accordance with the foregoing method described with respect to FIGS. 5A-5C provides a substantially cylindrical, circumferential chamber and avoids the presence of a seam that runs the length of the device. Whereas a device having a longitudinal seam may be prone to tearing and/or uneven inflation, the tubular structure of the present invention is believed to alleviate such problems. In addition, a device constructed in accordance with the present invention allows uniform patterns of contact points 22 to be formed between the outside and inside surfaces, thus enhancing uniformity of force distribution during expansion.

In operation, the deflated apparatus is inserted into the vaginal canal and positioned so that distal cuff 15 will engage the interior plane of the patient's uterus when deployed. Distal cuff 15 preferably expands as a cylinder near the patient's cervix, thereby preventing the lateral vaginal wall from converging. Proximal cuff 16 preferably provides a smooth lip that protects the patient's exterior vaginal area from unwanted contact such as rubbing or pinching during insertion and examination.

Advantageously, distal cuff 15 also may bias the patient's cervix towards the vaginal canal. Upon inflation, the apparatus forms a substantially cylindrical configuration having a lumen that permits viewing of, for example, the uterus. Once the exam is completed the inflatable body is allowed to deflate automatically by the force exerted by the cavity walls.

Referring again to FIG. 1, inflation lumen 17 extends along the entire length of inflatable body 11, and preferably is disposed between interior and exterior walls 20 and 21, respectively. Inflation lumen 17 may form a central spine that enhances the rigidity of the device in the deflated configuration, thus assisting insertion of the apparatus in the deflated configuration. In FIGS. 1-3 inflation lumen 17 illustratively is formed in a substantially helical configuration, e.g., a substantially cork screw shape. Alternatively, the inflation lumen may form a substantially straight line from the proximal cuff tQ the distal cuff. Of course, one of ordinary skill in the art will recognize that many other inflation lumen configurations are possible without departing from the scope of the present invention.

Inflation lumen 17 preferably fills the inflatable body from distal cuff 15 towards proximal cuff 16. This strategy for inflating the device to its expanded configuration tends to cause the apparatus to engage and lock itself into position within the vaginal canal during inflation. The apparatus utilizes a combination of the mechanical forces provided by inflation lumen 17 and inflatable body 11 working to provide a substantially cylindrically shaped scaffold. Alternatively, inflation lumen 17 may include a plurality of small holes along its length to provide more uniform inflation of inflatable body 11.

As described hereinabove, distal and proximal cuffs 15 and 16, respectively, are integrally formed from the single piece of tubular material used to form the main body portion. In alternative embodiments, however, the inflatable body, proximal cuff and distal cuff may be separately formed as individual sections and welded or glued together to form a composite of which portions are selectively inflated, thus permitting angulations and allowing articulation of the vaginal canal. This also may be accomplished by modifying the inflatable body to include individually inflatable sections operatively coupled to their own fill lumens.

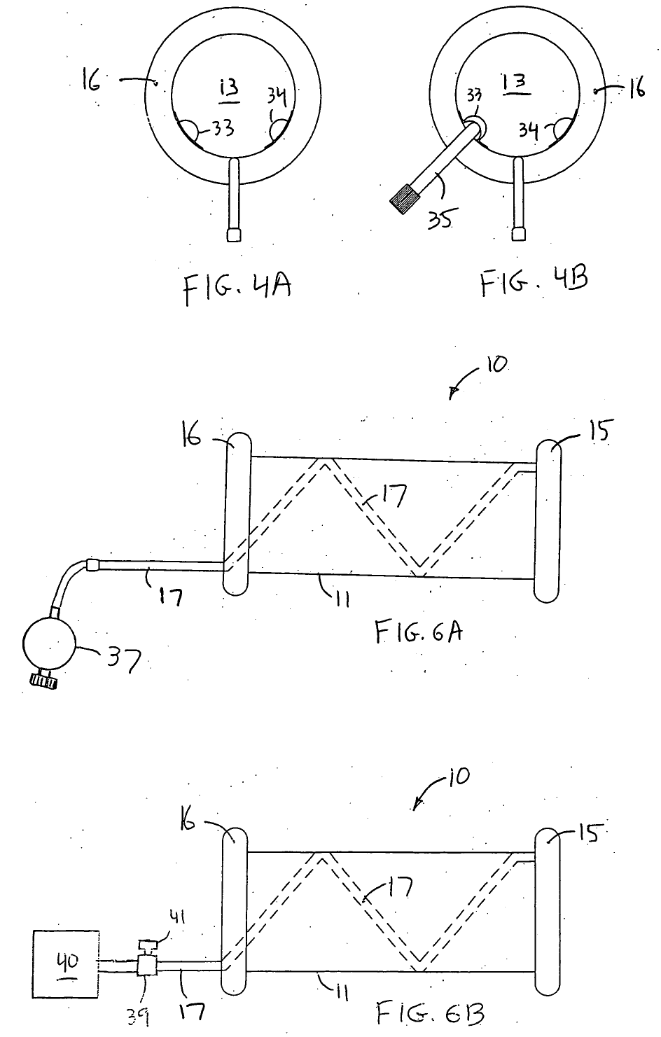

With respect to FIGS. 4A and 4B, central lumen 13 of inflatable body 11 includes channels 33 and 34 fitted along its length. Channels 33 and 34 may be used to secure tools 35, such as fiber optic light sources and suction/irrigation devices. Advantageously, the use of channels 33 and 34 frees up the physician's hands for other tasks.

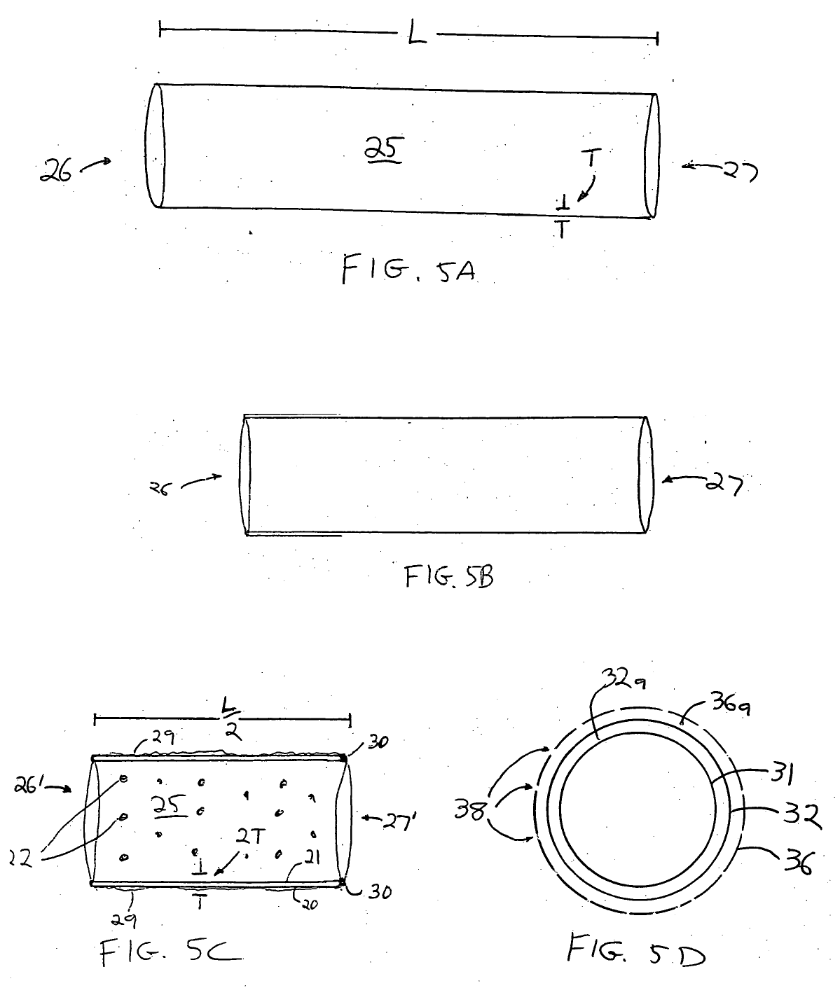

Referring now to FIGS. 6A and 6B, inflation lumen 17 is coupled to pump 37. Pump 37 provides a manually actuated source of pressurized fluid (e.g., water or saline) or gas (e.g., air) and may be actuated to inflate body 11. Alternatively, as depicted in FIG. 6B, inflation lumen 17 may be coupled to stopcock 39, which in turn is coupled to fluid source 40, such as an intravenous bag. Stopcock 39 includes knob 41 that may be actuated to modulate the influx of fluid or gas into inflatable body 11.

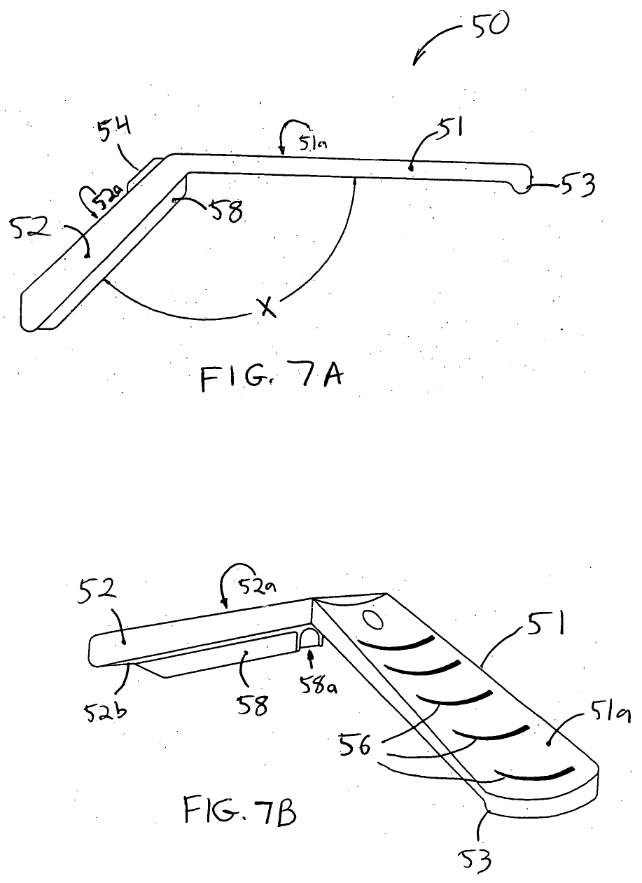

FIGS. 7-9 depict handle assembly 50 configured for use with inflatable body 11 described with respect to FIGS. 1-6. Handle assembly 50 includes intravaginal tongue portion 51 and gripping portion 52 for holding and manipulating the handle assembly. Tongue portion 51 includes anterior surface 51a, which is preferably concave to match the exterior contour of inflatable body 11. Handle assembly 50 facilitates insertion of the deflated device and manipulation of the internal anatomy of the vagina. Tongue portion 51 preferably includes distal lip 53 configured to engage the distal-end of a patient's vaginal canal such that the cervix can be manipulated and viewed.
Referring to FIG. 7A, tongue portion 51 is disposed at an angle X relative to gripping portion 52, thereby permitting a user to communicate substantial leverage to tongue portion 51 when-holding gripping portion 52. Preferably, angle X is greater than 90 degrees, more preferably between 120 and 160 degrees. Such an angle between the gripping and tongue portions facilitates insertion of the device and lessens the need to reposition the patient. Advantageously, this allows the patient to sit or lie in a more comfortable position during-most examinations.

According to some embodiments, gripping portion 52 includes thumb rest 54 designed to promote comfortable gripping of gripping portion 52 during a procedure. Thumb rest 54 is disposed generally on an anterior surface 52a of gripping portion 52 near the junction of the gripping and tongue portions. Thumb rest 54 preferably comprises a material that permits the handle assembly to be gripped securely while enhancing of the tactile sensation of the user. One suitable material is rubber.

Referring to FIG. 7B, according to some embodiments, the anterior surface of tongue portion 52 includes measurement indicia comprising radiopaque markings 56 that can be seen under fluoroscopic examination. Radiopaque markings 56 permit measurements to be taken with respect to surrounding objects such as organs, tumors, tissue and bones. By way of example, radiopaque markings 56 may be used to determine the depth or location of a tumor. Posterior surface 52b of gripping portion 52 includes guide 58 including substantially U-shaped channel 58a for securely supporting inflation lumen 17 during a procedure. Guide 58 may comprise a flexible material that allows the inflation lumen to be force-fit or snap-fit within U-shaped channel 58a.

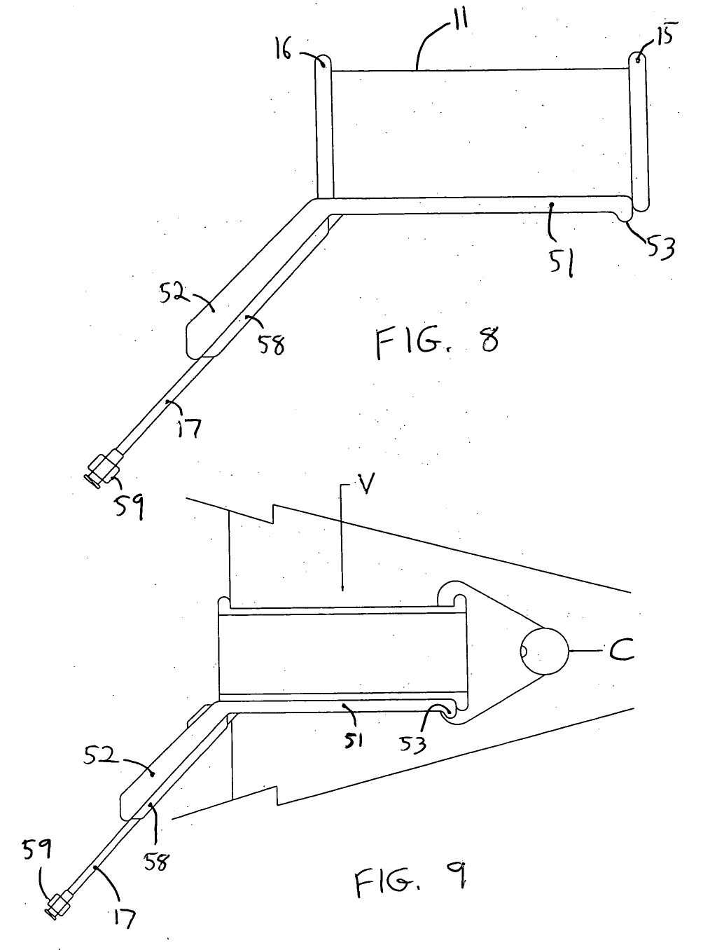

Referring to FIG. 8, inflatable body 11 is attached to the anterior surface of tongue portion 51, so that inflatable body 11 deploys outwardly from the anterior surface. Inflatable body 11 preferably is attached to tongue portion 51 using a suitable adhesive, ultrasonic welding or heat welding. Alternatively, the inflatable body may be attached to the tongue portion using a quick connector. In addition, inflation lumen 17 may be force-fit within flexible guide-58 and preferably includes connector 59, such as a conventional luer-type connector for attachment to pump 37 (FIG. 6A) or fluid source 40 (FIG. 6B). FIG. 8 depicts the inflatable body in the expanded configuration, wherein distal cuff 15 is disposed slightly distally with respect to distal lip 53.

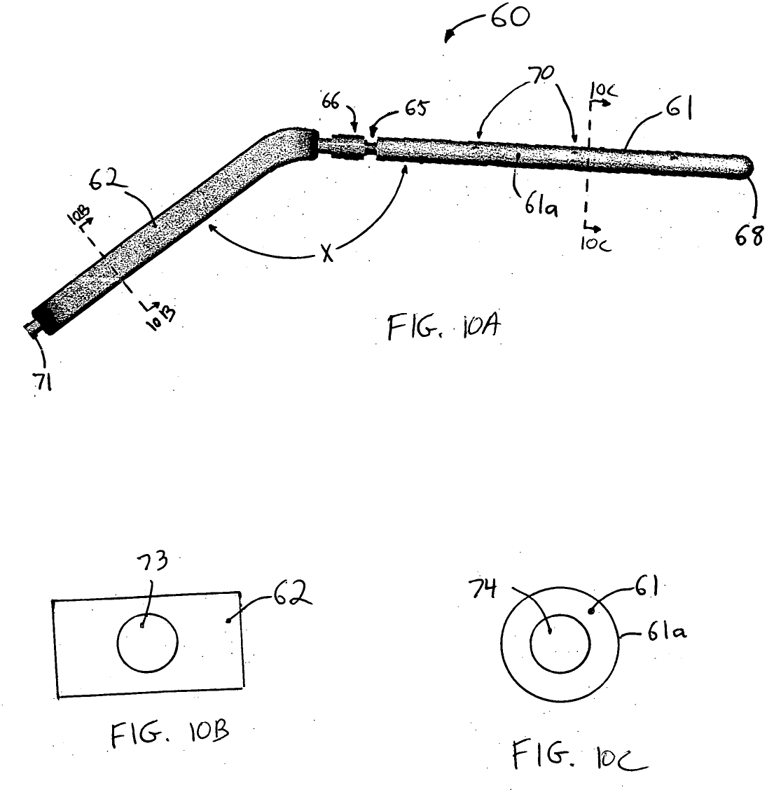

Referring now to FIG. 9, inflatable body 11 and handle assembly 50 are positioned within vaginal canal V of a patient. Distal lip 53 and distal cuff 15 preferably are configured to engage the distal end of vaginal canal V such that the patient's cervix C can be manipulated and viewed. Forces applied to the gripping portion by the physician produce resultant-forces applied to the inside of the terminus of the vaginal canal and subsequent anatomy. The proper manipulation of the gripping portion causes the cervix to present itself, thereby allowing the physician to view the junction between the cervix and vaginal canal at any desired angle.

FIGS. 10A-10C depict alternative handle assembly 60 adapted for use with inflatable body 11 of FIGS. 1-6. Handle assembly 60 is a modular assembly including intravaginal tongue portion 61 and detachable gripping portion 62. Preferably the tongue and gripping portions are releasably connected using a conventional luer type connectors 65 and 66, per se known in the art. More particularly, the proximal end of tongue portion 61 includes male luer component 65 adapted to mate with a corresponding female luer component 66 disposed on the distal end of gripping portion 62. These luer components preferably incorporate a standard twist-lock feature for engagement and disengagement. Alternatively, the tongue and gripping portions may be releasably connected by way of friction-fit, force-fit or snap-fit. Preferably the tongue and gripping portions are easy to attach and detach, but will not inadvertently detach during use.

As depicted in FIG. 10A, tongue portion 61 preferably includes convex anterior surface 61a, which is configured to match the interior contour of inflatable body 11. In the illustrated embodiment, tongue portion 61 is substantially cylindrical and anterior surface 61a includes one or more inflation holes 70 for expanding inflatable body 11. Gripping portion 62 includes a female luer component 71 configured to mate with a corresponding male luer component of an inflation device such as pump 37 (FIG. 6A) or fluid source 40 (FIG. 6B).

FIG. 10B is a cross-sectional view of the gripping portion of FIG. 10A taken along line 10B-10B and FIG. 10C is a cross-sectional view of the tongue portion of FIG. 10A taken along line 10C-10C. Gripping portion 62 includes lumen 73 that runs the length of the gripping portion and is in communication with the inflation device via connector 71. Although the cross-sec tion of the gripping portion is depicted as rectangular, it also may be other shapes including, but not limited to, square, circular, triangular and elliptical, without departing from the scope of the present invention.

With respect to FIG. 10C, tongue portion 61 also includes lumen 74 that runs the length of the tongue portion and is in communication with both lumen 73 and inflation holes 70. Although the cross-section of the gripping portion is depicted as circular, it also may be other shapes including, but not limited to, square, rectangular, triangular and elliptical, without departing from the scope of the present invention.

Referring again to FIG. 10A, tongue portion 61 is disposed at an angle X relative to gripping portion 62, thereby permitting a user to communicate substantial leverage to tongue portion 61 when holding gripping portion 62. Preferably, angle X is greater than 90 degrees, more preferably between 120 and 160 degrees. Such an angle between the gripping and tongues portions facilitates insertion of the device and lessens the need to reposition the patient. As noted hereinabove, this advantageously allows the patient to sit or lie in a more comfortable position during most examinations.

Similar to the previous embodiment, handle assembly 60 facilitates insertion of the deflated device and manipulation of the internal anatomy of the vagina. Tongue portion 61 includes distal tip 68 configured to engage the distal end of a patient's vaginal canal so that the cervix may be manipulated and viewed. The tongue portion provides for the manipulation of the patient's cervix by engaging the distal junction of the vaginal canal. Both the tongue and gripping portions preferably comprise hollow plastic pieces fabricated, e.g., using an injection molding process. Alternatively, the tongue and gripping portions may comprise other materials, such as metal or wood.

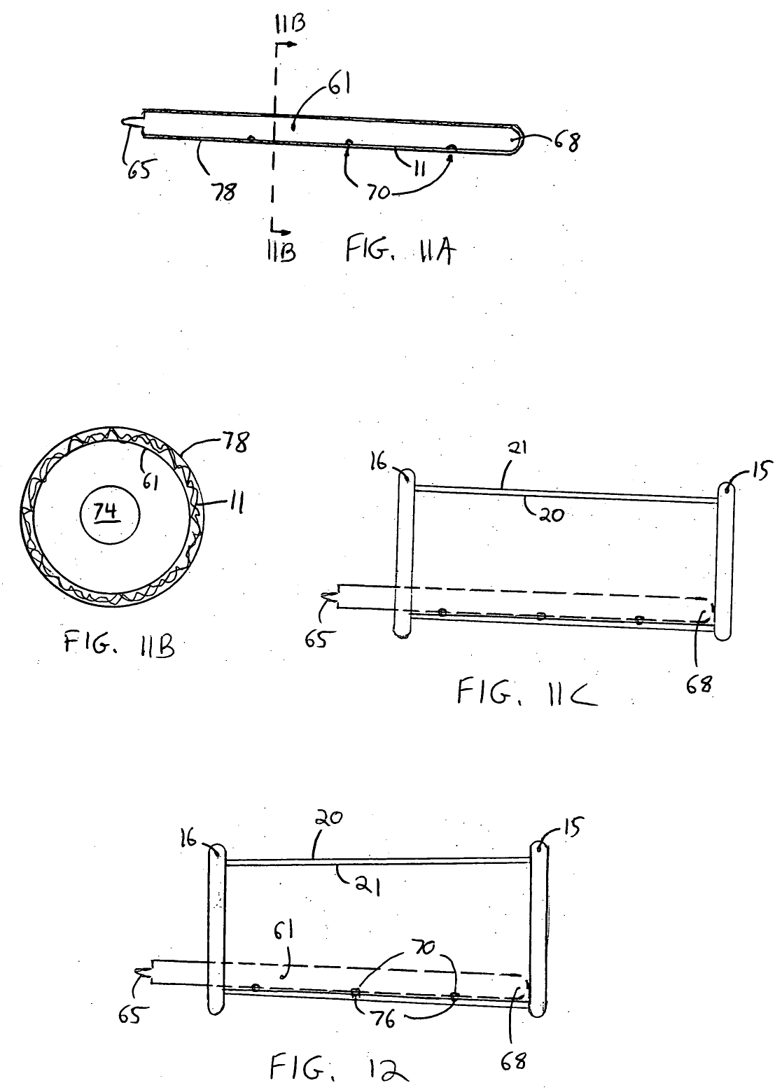

Referring to FIGS. 11A-11C, prior to insertion, the unexpanded inflatable body is attached to tongue portion 61 so that inflation holes 70 in tongue portion 61 are aligned with corresponding inflation holes 76 in interior wall 21 of inflatable body 11. Preferably, the tongue portion is disposed within the relatively soft inflatable body 11, thereby providing maximum comfort for the patient. Inflatable body 11 may be attached to tongue portion 61 using a suitable adhesive, ultrasonic welding or heat welding.

Sheath 78 is employed to facilitate insertion of the tongue portion and inflatable body. Sheath 78 is adapted to hold the inflatable body against the tongue portion during insertion. Additionally, sheath 78 is adapted to split open during expansion of inflatable body 11. Sheath 78 may include a series of perforations to facilitate splitting open during inflation. Sheath 78 may be removed by the physician after the procedure or, alternatively, may be configured to dissolve within the vaginal canal.

In another alternative embodiment depicted in FIG. 12, inflatable body 11 is tapered so that, in the expanded configuration, the distal end is larger in diameter than the proximal end. In addition, distal cuff 15 is larger in diameter than proximal cuff 16. This configuration assists in keeping the expanded inflatable body 11 in place within a patient's vaginal canal.

Preferably, a patient will be given the option of inserting the speculum by herself. Advantageously, it is easier for a patient to insert the tongue portion once the handle portion has been removed. In FIG. 11D, tongue portion 61 resembles a tampon having an elongated cylindrical surface and bullet-shaped distal tip 68. After insertion, the gripping portion optionally may be attached by way of the luer type connectors 65 and 66. In many instances, the cervix may be visualized without any manipulation of the handle assembly after insertion into the vagina. In these cases, there is no need to attach the gripping portion, as the female luer connector may be attached directly to the inflation device. However, in cases where the cervix cannot be visualized, the gripping portion may be attached to the tongue portion to provide the appropriate leverage for manipulating tongue portion 61.

Tongue portion 61 is designed to be thick enough to be usable as a lever without breaking, yet thin enough to provide comfort for the patient. Additionally, tongue portion 61 preferably is available in varying sizes for the treatment of different patients. Preferably, the tongue portion has a diameter of approximately 10 mm to 15 mm. Of course, as would be understood to those or ordinary skill in the art, the tongue portion may have a diameter other than 10 mm to 15 mm without departing from the scope of the present invention.

Inflatable body 11 preferably comes in multiple sizes, including a small size designed for young women and atrophic postmenopausal women, a medium size designed for “normal” women, and a large size designed for obese women. The small size preferably has a length of about 7.5 cm and a diameter of about 2.5 cm. The corresponding tapered design includes a diameter of about 2-cm near the proximal cuff and a diameter of about 2.5 cm near the distal cuff. The medium size preferably has a length of about 1.1 cm and a diameter of about 4.5 cm. The corresponding tapered design includes a diameter of about 3.5 cm near the proximal cuff and a diameter of about 5 cm near the distal cuff. The large size preferably has a length of about 16.5 cm and a diameter of about 6 cm. The corresponding tapered design includes a diameter of about 5 cm near the proximal cuff and a diameter of about 6.5 cm near the distal cuff.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

1. Apparatus for accessing a body cavity, comprising:
a tubular member formed by everting a length of material upon itself, the tubular member defining a main body portion;
a distal cuff disposed at a distal end of the main body portion; and.
a proximal cuff disposed at a proximal end of the main body portion,
wherein the tubular member has a deflated configuration for insertion into the body cavity and an expanded configuration when pressurized.

2. The apparatus of claim 1, wherein the main body portion forms an annulus having a central lumen.

3. The apparatus of claim 1, wherein the tubular member comprises an interior wall joined to one or more exterior walls at a plurality of contact points.

4. The apparatus of claim 3, wherein the contact points are arranged in a uniform pattern to enhance pressure distribution within the tubular member during inflation.

5. The apparatus of claim 1, wherein the proximal cuff and distal cuff are formed by locally produced chambers or features.

6. The apparatus of claim 1, wherein the distal cuff is configured to engage an interior plane of a patient's uterus.

7. The apparatus of claim 1., wherein the distal cuff is configured to prevent lateral portions of a patient's vaginal wall from converging.

8. The apparatus of claim 1, wherein the proximal cuff is configured to protect a patient's external vaginal area from rubbing or scraping.

9. The apparatus of claim 1, further comprising an inflation lumen that extends from the proximal cuff to the distal cuff.

10. The apparatus of claim 9, wherein the tubular member comprises an interior wall and an exterior wall and the inflation lumen is disposed between the interior wall and the exterior wall.

11. The apparatus of claim 9, wherein the inflation lumen enhances rigidity of the tubular member in the deflated configuration.

12. The apparatus of claim 9, wherein the inflation lumen has a substantially helical configuration.

13. The apparatus of claim 9, wherein the inflation lumen is configured to inflate the tubular member from the distal cuff to the proximal cuff.

14. The apparatus of claim 9, wherein the inflation lumen includes a plurality of holes that provide uniform inflation of the tubular member.

15. The apparatus of claim 2, further comprising a channel disposed within the central lumen.

16. The apparatus of claim 15, wherein the channel facilitates securing tools during a vaginal examination.

17. The apparatus of claim 1, wherein the tubular member is coupled to a pump configured to inflate the tubular member with gas or liquid.

18. The apparatus of claim 1, wherein the tubular member is coupled to a stopcock configured to modulate inflation of the tubular member.

19. The apparatus of claim 1, wherein the tubular member further comprises a lubricous exterior coating.

20. The apparatus of claim 1, wherein the tubular member further comprises a coating for topical distribution.

21. The apparatus of claim 20, wherein the coating contains drugs, genes or proteins.

22. The apparatus of claim 20, wherein the coating includes medications for treating yeast infections.

23. The apparatus of claim 1, wherein the tubular member further comprises an exterior layer including a plurality of micro-perforations for the passage of drugs in a liquid or gel form.

24. Apparatus for accessing a body cavity, comprising:
an inflatable body including a main body portion, a distal cuff disposed at a distal end of the main body portion and 4 proximal cuff disposed at a proximal end of the main body portion; and
a handle assembly attached to the inflatable body to facilitate insertion and manipulation of the inflatable body within the body cavity;
wherein the inflatable body has a deflated configuration for insertion into the body cavity and an expanded configuration when pressurized.

25. The apparatus of claim 24, wherein the handle assembly comprises an intravaginal tongue portion and a gripping portion for holding and manipulating the tongue portion.

26. The apparatus of claim 25, wherein the tongue portion includes a concave anterior surface to match an exterior contour of the inflatable body.

27. The apparatus of claim 25, wherein the tongue portion includes a distal lip that is configured to engage the distal end of a patient's vaginal canal.

28. The apparatus of claim 25, wherein the tongue portion is disposed at an angle relative to the gripping portion.

29. The apparatus of claim 28, wherein the angle is greater than 90 degrees.

30. The apparatus of claim 28, wherein the angle is between 120 degrees and 160 degrees.

31. The apparatus of claim 25, wherein the gripping portion includes a thumb rest.

32. The apparatus of claim 25, wherein an anterior surface of the tongue portion includes measurement indicia.

33. The apparatus of claim 32, wherein the scale comprises radiopaque markings.

34. The apparatus of claim 25, wherein the gripping portion further comprises a guide for supporting an inflation lumen.

35. The apparatus of claim 24, wherein the inflatable body is attached to the handle assembly using adhesives, ultrasonic welding or heat welding.

36. The apparatus of claim 24, wherein the inflatable body is coupled to a pump configured to expand the inflatable body with gas or liquid.

37. The apparatus of claim 24, wherein the inflatable body is coupled to a stopcock configured to modulate expansion of the inflatable body.

38. The apparatus of claim 24, wherein the inflatable body further comprises a lubricous exterior coating.

39. The apparatus of claim 24, wherein the inflatable body further comprises a coating for topical distribution.

40. The apparatus of claim 39, wherein the coating contains drugs, genes or proteins.

41. Apparatus for accessing a body cavity, comprising:
an inflatable body; and
a modular handle assembly attached to the inflatable body to facilitate insertion and manipulation of the inflatable body within the body cavity;
wherein the inflatable body has a deflated configuration for insertion into the body cavity and an expanded configuration when pressurized.

42. The apparatus of claim 41, wherein the modular handle assembly comprises an intravaginal tongue portion and a detachable gripping portion for holding and manipulating the tongue portion.

43. The apparatus of claim 42, wherein the tongue portion and gripping portion are releasably connected with a luer-type connector.

44. The apparatus of claim 42, wherein the tongue portion includes a convex anterior surface configured to mate with an interior surface of the inflatable body.

45. The apparatus of claim 42, wherein the tongue portion is substantially cylindrical.

46. The apparatus of claim 42, wherein the tongue portion includes at least one inflation hole for expanding the inflatable body.

47. The apparatus of claim 46, wherein the tongue portion includes a lumen in communication with the inflation holes.

48. The apparatus of claim 46, wherein the gripping portion includes a lumen in communication with the inflation holes.

49. The apparatus of claim 46, further comprising an inflation device in communication with the inflation holes.

50. The apparatus of claim 41, wherein the modular handle assembly comprises hollow plastic fabricated using an injection molding process.

51. The apparatus of claim 41, wherein the inflatable body is attached to the handle assembly using adhesives, ultrasonic welding or heat welding.

52. The apparatus of claim 41, further comprising a sheath that surrounds the inflatable body during insertion into the body cavity.

53. The apparatus of claim 41, wherein the sheath is adapted to burst open during expansion of the inflatable body.

54. The apparatus of claim 42, wherein the tongue portion is disposed at an angle relative to the gripping portion.

55. The apparatus of claim 54, wherein the angle is greater than 90 degrees.

56. The apparatus of claim 54, wherein the angle is between 120 degrees and 160 degrees.

57. The apparatus of claim 41, wherein the inflatable body is coupled to a pump configured to expand the inflatable body with gas or liquid.

58. The apparatus of claim 41, wherein the inflatable body is coupled to a stopcock configured to modulate expansion of the inflatable body.

59. The apparatus of claim 41, wherein the inflatable body further comprises a lubricous exterior coating.

60. The apparatus of claim 41, wherein the inflatable body further comprises a coating for topical distribution.

61. The apparatus of claim 60, wherein the coating contains drugs, genes or proteins.
